(19) United States
(12) Patent Application Publication
(10) Pub. No.: US 2014/0335142 A1
(43) Pub. Date: Nov. 13, 2014

(54) METHOD OF SURFACE TREATMENT OF AN IMPLANT, AN IMPLANT TREATED BY SAID METHOD AND AN ELECTROLYTE SOLUTION FOR USE IN SAID METHOD

(71) Applicants: Martina Frischherz, Ebikon (CH); Vinzenz Max Frauchiger, Solothurn (CH)

(72) Inventors: Martina Frischherz, Ebikon (CH); Vinzenz Max Frauchiger, Solothurn (CH)

(73) Assignee: SMITH & NEPHEW ORTHOPAEDICS AG, Memphis, TN (US)

(21) Appl. No.: 13/260,875

(22) PCT Filed: Apr. 2, 2009
(86) PCT No.: PCT/EP2009/002431

§ 371 Date: Jun. 20, 2014

(51) Int. Cl. C25D 9/08; A61L 31/16; A61L 31/08

(52) U.S. Cl. CPC C25D 9/08; A61L 31/086; A61L 31/16; A61L 31/088; A61L 2300/104; A61L 2300/606; A61L 2420/06; A61L 2420/02
USPC 424/423; 424/618; 205/318

(57) ABSTRACT

A method of surface treatment of at least part of an electro-conductive surface of an implant, in particular an orthopaedic or a dental implant is described which permits the simultaneous, electrochemical deposition of a therapeutic agent and a calcium phosphate coating in a combined single-step deposition process. The method involves the preparation of an electrolyte solution containing calcium and phosphorus ions and a therapeutic agent, preferably in combination with a complexing agent such that the resulting complex has a net positive charge. This electrolyte solution is then used in an electrochemical deposition process to produce a calcium phosphate coating incorporating the therapeutic agent on the electro-conductive surface of the implant Preferably, the therapeutic agent comprises metal ions, for example silver ions, and the complexing agent comprises an ammine complex. Also provided are an implant treated by the described method and an electrolyte solution for use in the described method.

The present invention relates to a method of surface treatment of an implant, an implant treated in accordance with this method and an electrolyte solution for use in the method.

Implant-related infection following joint replacement is a serious complication in orthopaedic surgery and in extreme cases may require removal of the prosthesis. Infections develop primarily because the interface between artificial implant and tissue is not as well supplied with blood as other parts of the tissue so that parenteral drug administration may not be effective. Antibiotics have to be applied locally to fight infection and to kill the pathogenic germs completely. In the past this has been attempted by bonding antimicrobials directly to the surface of the implant or by incorporating conventional antibiotics into resorbable polymer coatings, allografts, acrylic cements, or calcium phosphate cements. However, these methods tend to impede good osteointegration of the artificial implant surface.

More recently, calcium phosphate implant coatings with antimicrobial properties have been produced by the incorporation of silver ions into the coating. Calcium phosphate coatings, which mostly consist of hydroxyapatite, allow for direct and chemical bone bonding to the artificial implant surface. One method of depositing such coatings onto implant surfaces is a plasma spray technique. However, if silver ions have to be included into the plasma sprayed coatings, this is time consuming and sprayed coatings can only be applied to surfaces of the implant in the line of sight, which is a major disadvantage of this method. Another method is via an electrochemical (i.e. cathodic) deposition of brushite on to the implant but a drawback of electrochemically deposited coatings is their limited mechanical integrity. On the other hand, this method has the advantage that it can be applied to any conductive implant grade substrate. However, in order to incorporate silver ions into such coatings a post treatment has to be applied whereby an ion exchange reaction is produced in a silver nitrate solution. Yet another method is to use sol/gel coatings which are applied to the implant using simple dipping techniques but in order to form a dense gel such coatings have to undergo a heat treatment that may have detrimental effects on the substrate.

An object of the present invention is to provide a method of surface treatment of an implant and thereby an implant treated by this method which overcomes the aforementioned disadvantages by simultaneously electrochemically depositing a therapeutic agent and a calcium phosphate coating in a combined single-step deposition process.

According to a first aspect of the present invention there is provided a method of surface treatment of at least part of an electro-conductive surface of an implant comprising the steps of
preparing an electrolyte solution containing calcium and phosphorus ions and a therapeutic agent; and
electrochemically depositing a calcium phosphate coating incorporating said therapeutic agent on said electro-conductive surface of the implant.

In a preferred embodiment, the electrolyte solution contains in combination with the therapeutic agent a complexing agent such that the resulting complex has a net positive charge.

In the present invention, incorporation of the therapeutic agent into the calcium phosphate coating takes place during the electrochemical deposition of the coating. As the calcium phosphate coating and the therapeutic agent are deposited simultaneously the number of steps required in the manufacture of an implant is reduced. In contrast to conventional manufacturing processes, the present invention reduces the number of steps required to produce a suitable coating on the implant to a single step process. It will be appreciated, however, that multiple coating steps may be used to create tailored and time-dependent release of therapeutic agents.

Preferably, the therapeutic agent comprises an antimicrobial agent. Such an antimicrobial agent may comprise a metallic, an inorganic, organic or biological antimicrobial agent. Advantageously, the antimicrobial agent comprises a metal ion, in particular silver, copper, zinc or cobalt ions or a mixture of same.

In general, metal phosphates exhibit a low solubility in water at neutral to alkaline pH values. This is especially true for metals which are of interest for as antimicrobial agents, such as copper, silver and cobalt. If minute amount of these metals, as ions in the form of a metal salt, are added to a calcium phosphate solution an immediate precipitation reaction can be observed and the meant-to-be antimicrobial agent may be no longer available for deposition on an implant surface via electrochemical deposition. The use of a complexing agent overcomes this problem by keeping the metal ions in solution, for example as a positively charged ammine-complex. In addition to stabilization of the solution, this kind of complex has the advantage of being positively charged and hence is still electrostatically attracted by a negatively polarized cathode formed by the electro-conductive surface of the implant.

Preferably also, therefore, the complexing agent comprises an (di)-ammine complex building agent. Other complex forming molecules may, however, be used provided that the resulting complex has a net positive charge.

Calcium phosphates tend to precipitate in alkaline solutions whilst, for example, a silver diammin complex starts to dissociate at pH values below 6.

Preferably, therefore, during preparation of the electrolyte solution a base solution is formed prior to addition of the complexing agent that has a pH value between 7 and 9 inclusive.

As the invention does not necessarily involve any thermal post-treatment of the implant and takes place in a very moderate chemical and electrochemical environment, it is possible to include further actives such as organic or biologic, for example peptides, DNA, etc., molecules in the electrolyte solution provided that they produce the prerequisite of a positive charge in solution.

According to a second aspect of the present invention there is provided an implant with an at least partial electro-conductive surface which has undergone a surface treatment of at least part of said electro-conductive surface of an implant, the surface treatment comprising the steps of
preparing an electrolyte solution containing calcium and phosphorus ions and a therapeutic agent; and
electrochemically depositing a calcium phosphate coating incorporating said therapeutic agent on said electro-conductive surface of the implant.

In a preferred embodiment, the electrolyte solution contains in combination with the therapeutic agent a complexing agent such that the resulting complex has a net positive charge.

Preferably, the implant has an electro-conductive surface formed by a titanium alloy.

Preferably also, the titanium alloy comprises Ti-6Al-7Nb, Ti-6Al-4V or commercially pure Ti.

In an alternative embodiment the implant has an electro-conductive surface formed by a zirconium alloy, in particular Zr-2.5Nb.

According to a third aspect of the present invention there is provided an electrolyte solution for use in the surface treatment of at least part of an electro-conductive surface of an implant comprising calcium and phosphorus ions and a therapeutic agent.

In a preferred embodiment a therapeutic agent is used in combination with a complexing agent such that the resulting complex has a net positive charge.

Further preferred but non-essential features of the various aspects of the present invention are described in the dependent claims appended hereto.

The various aspects of the present invention will now be further described by way of example.

In order to simultaneously, electrochemically deposit a therapeutic agent such as silver ions and a calcium phosphate coating in a combined single-step deposition process it is first necessary to prepare an electrolyte solution containing silver, calcium and phosphate ions. This can be achieved as follows.

First, a 1M ammonia solution is prepared to form the base electrolyte. The initial pH value of this solution is approximately 11 to 12 and must be reduced by adding 10M nitric acid until it is approximately 7.8 for the reasons indicated above. A predetermined amount of silver nitrate (AgNO₃) is then added. This amount may comprise, for example, 0.02 g/l but up to ten times this quantity is possible if required, i.e. the predetermined amount may be between 0.01 g/l and 0.20 g/l inclusive. As indicated above, other metal salts can be used instead of or in addition to silver nitrate dependent on the metal ions it is desired to deposit on the implant.

After complete dissolution of the metal salt, ammonium phosphate and calcium nitrate are added to the electrolyte solution. Preferably, the Ca/P-ratio is 1.0.-2.0, more preferably 1.2-1.8 and even more preferably 1.67 and the concentrations of calcium and phosphate are Calcium: 0.042 M and Phosphate: 0.025 M. The resulting electrolyte solution has a pH value before the start of the electrochemical deposition between 5.5 and 4.5 inclusive and stabilizes at 4.5.

Surface treatment of the implant can now take place using cathodic deposition to deposit a silver-containing calcium phosphate layer on the implant surface. Preferably, the implant or at least an electro-conductive surface of same comprises a titanium alloy, for example Ti-6Al-7Nb, which has been slightly etched in HF/HNO₃-solution prior to the electrochemical coating procedure. Alternatively, the implant or at least an electro-conductive surface of same comprises a titanium alloy in form of Ti-6Al-4V or commercially pure Ti. In a further alternative embodiment, the implant or at least an electro-conductive surface of same comprises a zirconium alloy, in particular in form of Zr-2.5Nb.

In one embodiment of the invention, during the electrochemical coating procedure the voltage used is between 1 and 3 V inclusive in potentiostatic mode. In another embodiment of the invention, during the electrochemical coating procedure the electric current used is between 0.001 and 0.2 mA/cm² inclusive in galvanostatic mode. Particularly preferred is an electric current of approximately 0.01 mA/cm². A graphite or titanium anode is also used and the electrolyte solution is constantly stirred using a magnetic stirrer. A typical process duration is 30 minutes but this is dependent on the desired coating thickness and structure. During the deposition process the pH of the solution changes to slightly higher values.

After treatment, the silver contained in the coating on the implant is at least partly of a metallic nature. Moreover, the base colour of the coating is grey to black dependent on the silver concentration used in the electrolyte solution.

As indicated previously, other or additional metal ions can be used as therapeutic agents, for example ions of zinc, copper, cobalt, aluminium can be used Likewise, other complexing agents can be used to stabilize these metal ions in the electrolyte solution, for example an ethylenediamine complex.

In addition to metal ions, other antimicrobial actives such as antibiotics, antiviral drugs or fungicides may be added in an appropriate form to the electrolyte solution provided that these agents are positively charged in the electrolyte solution. Similarly, other therapeutic agents such as growth factors, bisphosphonates, peptides, DNA etc., can be used. It will thus be appreciated that an implant can be treated to produce multi-layer systems creating tailored release profiles of a variety of actives. Also, after treatment according to the invention additional actives can be incorporated into the already deposited layer using electrophoresis or similar techniques. To achieve this, the deposited calcium phosphate layer may be heat treated prior to the deposition of additional actives. A typical heat treatment is performed in a vacuum or inert gas furnace at 550° C./1 h with appropriate heating and cooling rates.

It will be appreciated that the method of treatment of an implant in accordance with the present invention has several advantages over the prior art. First, the incorporation of a therapeutic agent in a calcium phosphate coating is possible using a simple single step coating process. No ion exchange reaction is needed and common chemicals can be used as electrolyte constituents. It is believed that in comparison to sol/gel coatings, the method of the present invention does not require any subsequent heat treatment to finalize the coating properties.

1. A method of surface treatment of at least part of an electro-conductive surface of an implant comprising the steps of:
preparing an electrolyte solution containing calcium and phosphorus ions and a therapeutic agent; and
electrochemically depositing a calcium phosphate coating incorporating said therapeutic agent on said electro-conductive surface of the implant.

2. A method as claimed in claim 1, wherein the electrolyte solution comprises in combination with a therapeutic agent a complexing agent such that the resulting complex has a net positive charge.

3. A method as claimed in claim 1 or 2, wherein the therapeutic agent comprises an antimicrobial agent.

4. A method as claimed in claim 3, wherein the antimicrobial agent comprises an inorganic, organic or biological antimicrobial agent.

5. A method as claimed in any of claims 1 to 4, wherein the therapeutic agent comprises metal ions.

6. A method as claimed in claim 5, wherein the therapeutic agent comprises silver, copper, zinc or cobalt ions or a mixture of same.

7. A method as claimed in any of claims 1 to 6, wherein the complexing agent comprises a (di-) ammine complex building agent.

8. A method as claimed in any of claims 2 to 7, wherein during preparation of the electrolyte solution a base solution is formed prior to addition of the complexing agent that has a pH value between 7 and 9 inclusive.

9. A method as claimed in any of claims 1 to 8, wherein the preparation of the electrolyte solution comprises the following steps:
preparing an ammonia solution to form a base electrolyte;
reducing the pH value of the ammonia solution to 8 or less by the addition of an acid thereto;
adding a predetermined quantity of a metal salt thereto; and
adding ammonium phosphate and calcium nitrate thereto after dissolution of the metal salt.

10. A method as claimed in claim 9, wherein the metal salt comprises a silver salt.

11. A method as claimed in claim 9 or claim 10, wherein the metal salt is silver nitrate.

12. A method as claimed in any of claims 9 to 11, wherein the predetermined quantity of the metal salt is between 0.01 g/l and 0.20 g/l inclusive.

13. A method as claimed in any of claims 9 to 12, wherein the pH value of the ammonia solution is reduced by the addition of a mineral or organic acid thereto.

14. A method as claimed in claim 13, wherein the pH value of the ammonia solution is reduced by the addition of nitric acid, hydrochloric acid, phosphoric acid, and/or acetic acid thereto.

15. A method as claimed in any of claims 9 to 14, wherein the Ca/P-ratio in the electrolyte solution is 1.0-2.0, preferably 1.2.-1.8.

16. A method as claimed in any of claims 9 to 15, wherein the pH value of the electrolyte solution is between 7.5 and 4.5 inclusive.

17. A method as claimed in any of claims 1 to 16, wherein during the electrochemical deposition of the calcium phosphate coating a voltage between 1 V and 3 V inclusive in potentiostatic mode is used.

18. A method as claimed in any of claims 1 to 17, wherein during the electrochemical deposition of the calcium phosphate coating an electric current between 0.001 and 0.2 mA/cm² inclusive in galbvanostatic mode is used.

19. A method as claimed in any of claims 1 to 18, wherein during the electrochemical deposition of the calcium phosphate coating the electrolyte solution is stirred using a magnetic stirrer.

20. A method as claimed in any of claims 1 to 19, wherein the electrochemical deposition process has a duration of the order of 10-120 minutes, preferably 15-60 minutes and more preferably 20-45 minutes.

21. A method as claimed in any of claims 2 to 20, wherein the complexing agent comprises an ethylenediamine.

22. A method of Manufacturing an implant comprising the method of any one of the preceding claims.

23. An implant with an at least partial electro-conductive surface which has undergone a surface treatment of at least part of said electro-conductive surface of an implant, the surface treatment comprising the steps of:
preparing an electrolyte solution containing calcium and phosphorus ions and a therapeutic agent; and
electrochemically depositing a calcium phosphate coating incorporating said therapeutic agent on said electro-conductive surface of the implant.

24. An implant according to claim 23, whereby the surface treatment comprises the step of preparing the electrolyte solution containing calcium and phosphorus ions and a therapeutic agent in combination with a complexing agent such that the resulting complex has a net positive charge.

25. An implant as claimed in claim 23 or 24, wherein said electro-conductive surface is formed by a titanium alloy.

26. An implant as claimed in claim 25, wherein said titanium alloy comprises Ti-6Al-7Nb, commercially pure Ti, and/or Ti6Al4V.

27. An implant as claimed in claim 23 or 24, wherein said electro-conductive surface is formed by a zirconium alloy, in particular Zr-2.5 Nb.

28. An implant as claimed in any of claims 20 to 27, comprising an orthopaedic or a dental implant

29. An electrolyte solution for use in the surface treatment of at least part of an electro-conductive surface of an implant comprising calcium and phosphorus ions and a therapeutic agent.

30. An electrolyte solution according to claim 29, comprising in combination with the therapeutic agent a complexing agent such that the resulting complex has a net positive charge.

31. A solution as claimed in claim 29 or 30, wherein the therapeutic agent comprises an antimicrobial agent.

32. A solution as claimed in any of claims 29 to 31, wherein the therapeutic agent comprises metal ions.

33. A solution as claimed in claim 32, wherein the therapeutic agent comprises silver, copper, zinc or cobalt ions or a mixture of same.

34. A solution as claimed in any of claims 30 to 33, wherein the complexing agent comprises a (di-)ammine complex building agent.

35. A solution as claimed in any of claims 29 to 34, which comprises an ammonia solution.

36. A solution as claimed in any of claims 29 to 35, produced by the following steps:
preparing an ammonia solution to form a base electrolyte;
reducing the pH value of the ammonia solution to less than 9 by the addition of an acid thereto;
adding a predetermined quantity of a metal salt thereto; and
adding a soluble phosphate and a calcium salt thereto after dissolution of the metal salt.

37. A solution as claimed in any of claims 32 to 36, wherein the metal salt comprises a silver salt.

38. A solution as claimed in any of claims 32 to 37, wherein the metal salt is silver nitrate.

39. A solution as claimed in any of claims 32 to 38, wherein the predetermined quantity of the metal salt is between 0.01 g/l and 0.20 g/l inclusive.

40. A solution as claimed in any of claims 36 to 39 wherein the acid is nitric acid.

41. A solution as claimed in any of claims 29 to 40, wherein the Ca/P-ratio is 1.0-2.0, preferably 1.2-1.8.

42. A solution as claimed in any of claims 29 to 41, which has a pH value between 7.5 and 4.5 inclusive.
